(19) United States
(12) Patent Application Publication
(10) Pub. No.: US 2024/0382150 A1
(43) Pub. Date: Nov. 21, 2024

(54) A MUSCLE SPASTICITY MEASUREMENT SYSTEM AND SENSOR

(71) Applicant: NANYANG TECHNOLOGICAL UNIVERSITY, Singapore (SG)

(72) Inventors: Pooi See LEE, Singapore (SG); Kaushik PARIDA, Singapore (SG); Dace GAO, Singapore (SG)

(21) Appl. No.: 18/557,115

(22) PCT Filed: May 17, 2022
(86) PCT No.: PCT/SG2022/050325

§ 371 Date: Oct. 25, 2023

(30) Foreign Application Priority Data
May 17, 2021 (SG) 10202105124Y

(51) Int. Cl. A61B 5/00

(52) U.S. Cl. CPC A61B 5/4519

(57) ABSTRACT

A muscle spasticity measurement system and sensor comprise a housing having a flexible wall and a constraining wall defining a cavity. A piezoelectric device is disposed in the cavity between an actuating bag and the flexible wall. The piezoelectric device is coupled to the housing substantially via an interior face of the flexible wall. The actuating bag is configured to be pneumatically operable. The actuating bag in an inflated state is constrainable by the housing to press an exterior face of the flexible wall against a body part of the subject.

The present application claims priority to the Singapore patent application no. 10202105124Y, the entire disclosure of which is incorporated herein by reference.

### TECHNICAL FIELD

The present disclosure relates to the field of measuring for diagnostic and other purposes, and more particularly to a muscle spasticity measurement system and sensor.

### BACKGROUND

Spasticity is the stiffness of muscles prevalent in patients suffering from stroke, cerebral palsy, and spinal cord injury. It is the disorder associated with the central nervous system which causes impairment of the patient's function, impairment of mobility, pain, joint contractures, and pressure ulcers. Currently, the intensity of spasticity is a scoring on a six-point “Modified Ashworth Scale”, and entirely based on the clinician's sense of touch. The scoring is highly subjective and can be inconsistent. Moreover, mild levels of spasticity may not be detected by the conventional method.

### SUMMARY

In one aspect, the present application discloses a sensor attachable to a subject. The sensor comprises a housing having a flexible wall and a constraining wall, the housing defining a cavity; an actuating bag disposed in the cavity; and a piezoelectric device disposed in the cavity between the actuating bag and the flexible wall, the piezoelectric device being coupled to the housing substantially via an interior face of the flexible wall, wherein the actuating bag is configured to be pneumatically operable, and wherein the actuating bag in an inflated state is constrainable by the housing to press an exterior face of the flexible wall against a body part of the subject. Optionally, the cavity is characterized by a fixed volume. Optionally, the cavity is characterized by a closed volume. The sensor is preferably configured to measure a level of muscle stiffness at the body part. The level of muscle stiffness preferably corresponds to a muscle stiffness of a bicep of the subject.

The actuating bag may be characterized by a variable bag volume, the actuating bag being pneumatically operable to increase the variable bag volume until the variable bag volume is constrained by the housing. The actuating bag may be pneumatically operable to expand until the actuating bag is constrained by the constraining wall. The actuating bag may be pneumatically operable to increase in the variable bag volume until the actuating bag is constrained by a pressure in the cavity.

The flexible wall may comprise a first surface and a substantially opposing second surface, and wherein the second surface is a part of an external surface of the sensor. The flexible wall may be resiliently deformable to vary a shape of the cavity.

The piezoelectric device may be configured to provide a signal corresponding to a measurement state of the sensor, wherein in the measurement state the actuating bag is in abutment with the constraining wall and the piezoelectric device. The flexible wall may be in abutment with the body part.

The sensor may be configured such that the actuating bag is configured to expand to a threshold pressure and sustain the actuating bag at a substantially constant pressure in the measurement state. The sensor may be configured to expand to a threshold volume and sustain the actuating bag at a substantially constant volume in the measurement state.

The sensor is configured such that the piezoelectric device comprises a piezoelectric film.

In another aspect, a muscle spasticity measurement system comprising: the sensor according to any described above; a controller operably coupled to the sensor, the controller being configured to receive a signal from the piezoelectric device and to determine a muscle stiffness value based on the signal. The controller may be configured to controllably expand the actuating bag in the cavity. The controller may be configured to: expand the actuating bag to a threshold pressure; and hold the actuating bag at the threshold pressure.

The muscle spasticity measurement system in which the controller is further configured to: receive the signal produced by the piezoelectric device when the actuating bag is at the threshold pressure; and determine the muscle stiffness value based on the signal. The controller is configured to expand the actuating bag to a threshold volume; and to hold the actuating bag at the threshold volume.

The controller may be further configured to: receive the signal produced by the piezoelectric device when the actuating bag is at the threshold volume; and determine the muscle stiffness value based on the signal. The muscle spasticity measurement system in which the controller may be configured to acquire a plurality of the signal over time, and wherein the controller is configured to determine a mean muscle stiffness value at the body part based on the plurality of the signal.

### BRIEF DESCRIPTION OF DRAWINGS

FIGS. 1A and 1B are schematic diagrams of a sensor according to an embodiment of the present disclosure.

FIG. 2 is a sectional view of another embodiment of the sensor in an initial state.

FIG. 3 is a schematic diagram of the piezoelectric device of the sensor of FIG. 2.

FIG. 4 is a sectional view of the sensor of FIG. 2 in a measurement state.

FIG. 5 is a schematic diagram of the piezoelectric device of the sensor of FIG. 4.

FIG. 6 is a sectional view of another embodiment of the sensor.

FIG. 7 is a schematic diagram of a muscle spasticity measurement system according to an embodiment of the present disclosure.

FIG. 8 illustrates an embodiment of the sensor used in measuring muscle spasticity of a subject's bicep.

FIG. 9 illustrates an embodiment of the sensor as a wearable product.

FIG. 10 illustrates exemplary embodiments in which the sensor is attachable to a body part.

FIG. 11 is a flowchart of a method of measuring muscle spasticity according to an embodiment of the present disclosure.

FIG. 12 is a stress-strain plot of the porcine muscle kept at room temperature measured using a conventional mechanical stress-strain experimental setup.

FIG. 13 is a stress-strain plot of the porcine muscle kept at minus-20° C. (20 degrees Celsius below zero) for one hour, measured using a conventional mechanical stress-strain experimental setup.

FIG. 14 is a voltage response (in wavelet form) of the signal received from the measurement system 100 when measuring a stiffness of the porcine muscle kept at room temperature.

FIG. 15 is a voltage response (in wavelet form) of the signal received from the measurement system 100 used to measure a stiffness of the porcine muscle kept at minus-20 degrees Celsius for one hour.

FIG. 16 is a response of the measurement system 100 as a function of the stiffness of porcine muscle.

FIG. 17 is a voltage response (in wavelet form) of the signal received from the measurement system 100 used to measure a low stiffness bicep muscle of a human subject.

FIG. 18 shows a voltage response (in wavelet form) of the signal received from the measurement system 100 used to measure a high stiffness bicep muscle of a human subject.

### DETAILED DESCRIPTION

Reference throughout this specification to “one embodiment”, “another embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment. Furthermore, the described features, structures, or characteristics may be combined in any suitable manner in one or more embodiments. In the following description, numerous specific details are provided to give a thorough understanding of embodiments. One skilled in the relevant art will recognize that the various embodiments may be practiced without one or more of the specific details, or with other methods, components, materials, etc. In other instances, some or all known structures, materials, or operations may not be shown or described in detail to avoid obfuscation.

The word “exemplary” is used herein to mean “serving as an example, instance, or illustration.” Any embodiment described herein as “exemplary” is not necessarily to be construed as preferred or advantageous over other embodiments. As used herein, the singular ‘a’ and ‘an’ may be construed as including the plural “one or more” unless apparent from the context to be otherwise.

Terms such as “first” and “second” are used in the description and claims only for the sake of brevity and clarity, and do not necessarily imply a priority or order, unless required by the context. The terms “about” and “approximately” as applied to a stated numeric value encompasses the exact value and a reasonable variance, as will be understood by one of ordinary skill in the art, and the terms “generally” and “substantially” are to be understood in a similar manner, unless otherwise specified.

As will be evident from the various non-limiting examples below, the present disclosure is applicable in a wide variety of situations, including but not limited to measuring a level of muscle spasticity of a body part of a subject without causing any harm/damage to the body part. In addition to being diagnostic tools for determining abnormal or medical conditions of muscle spasticity, various embodiments of the measuring system 100 and/or sensor 200 disclosed herein may be used with subjects with no known medical conditions of muscle spasms, e.g., in sports science, physiotherapy, etc., where the sensing or measuring of muscle conditions may be useful. The terms “spasticity” and “muscle stiffness” are used interchangeably in the present disclosure in referring to muscle tightness, including but not limited to normal muscle contractions, abnormal muscle tightness due to prolonged muscle contraction, sudden and involuntary contractions of one or more muscles (cramps), etc.

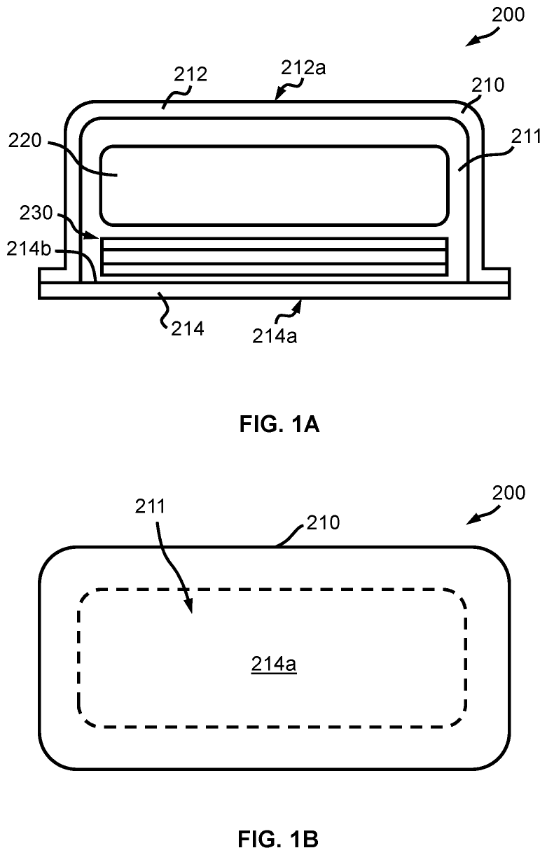

FIG. 1A is a schematic cross-sectional view of a sensor 200 according to one embodiment of the present disclosure. The sensor 200 is also referred to as a soft-pressure sensor-actuator-strain-measuring (SPASM) device in the present disclosure. The sensor 200 includes an actuating bag 220 and a piezoelectric device 230 disposed in a housing 210.

The housing 210 includes a constraining wall 212 and a flexible wall 214. At least a part of the constraining wall 212 and at least a part of the flexible wall 214 are spaced apart. Optionally, a part of the constraining wall 212 and a part of the flexible wall 214 are substantially opposite one another, with an actuating bag 220 and a piezoelectric device 230 disposed between the part of the constraining wall 212 and the part of the flexible wall 214. In some embodiments, the constraining wall 212 and the flexible wall 214 define a substantially fixed spacing therebetween (e.g., by the configuration, shape, nature and/or amount of material that forms the housing 210) such that the cavity 211 is essentially characterized by a fixed volume. The cavity 211 may also be concurrently characterized as a closed volume in that the actuating bag 220 cannot escape or move out of the cavity 211. The flexible wall 214 includes a first surface (interior face 214b) and a substantially opposing second surface (exterior face 214a). The second surface (exterior face 214a) is a part of an external surface of the sensor 200. A planar area of the external face 214a may be substantially flat when not in use or it may be preconfigured with a curvature.

The housing 210 is preferably soft or flexible. The housing 210 may be made of one flexible material, or a composite of at least one flexible material, such as one selected from fabrics, fabric-like materials, nonwovens, polymeric films, etc. The constraining wall 212 may be formed from a flexible material such that it is helps to define a cavity 211 of a variable shape. The flexible wall 214 is configured to be soft or flexible such that when the flexible wall is pressed against an external surface (e.g., a subject's skin near the muscle to be measured), the flexible wall 214 may conform to the shape of the external surface. For example, the flexible wall may be resiliently deformable to vary a shape of the cavity 211.

The constraining wall 212 is preferably configured to be sufficiently flexible to an extent such that a central part of the housing 210 or the sensor 200 may conform to the shape of the external surface against which the housing 210 or the sensor 200 is disposed. For example, if the external surface is part of a subject's arm, the external surface would have a certain curvature, and the housing 210 is preferably sufficiently flexible for the sensor 200 to be follow the curvature of the arm such that the external surface closely contacts the arm. When the sensor 200 is in use, an exterior face 214a of the flexible wall 214 is in direct contact with the external surface. The constraining wall 212 and the flexible wall 214 may be made of similar materials or dissimilar materials. Each or both of constraining wall 212 and the flexible wall 214 may be made of more than one layer of materials. Preferably, the exterior face 214a of the flexible wall 214 is made of a material that is comfortable and suitable for direct contact with the subject's skin. When the sensor 200 is in use, the sensor is disposed on the subject with an external face 212a of the constraining wall 212 facing away from the flexible wall 214.

In some examples, the constraining wall 212 and the flexible wall 214 are contiguously or integrally formed to define a cavity 211 in the housing 210. In some other examples, the constraining wall 212 and the flexible wall 214 are joined together to form a cavity 211. In some examples, the sensor 200 may be formed by a process of disposing the piezoelectric device 230 on the flexible wall 214, disposing the actuating bag 220 on the piezoelectric device 230, and layering the constraining wall 212 on the actuating bag 220. As shown in FIG. 1B, the constraining wall 212 is directly or indirectly joined or integrally formed with the flexible wall 214 around a perimeter such that the cavity 211 (hidden and indicated by a dashed line) forms a closed volume or a substantially enclosed space, with an external face 214a of the flexible wall 214 presenting a planar area for contacting the subject.

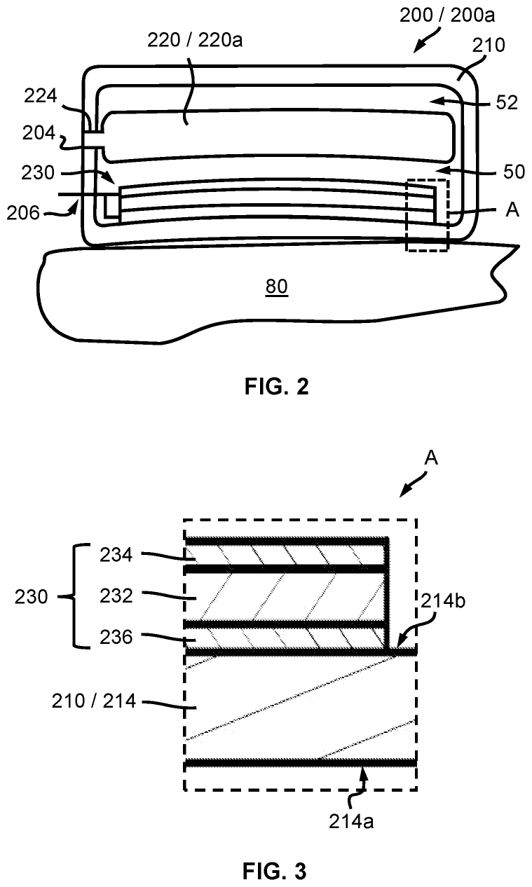

In the present disclosure, the term “cavity” refers to the interior space of the housing 210. The cavity 211 may be described as a “closed volume”, i.e., the cavity 211 provides a volumetric space that is substantially enclosed such that actuating bag 220 (disposed in the cavity 211) cannot be partially or fully outside the cavity 211. For example, the cavity 211 may be described as a closed cavity in that the cavity 211 is substantially enclosed by the housing 210 on all sides. Non-fluid contents of the cavity 211 are prevented from being partially or fully displaced from the inside of the housing 210 to the outside of the housing 210. Examples of non-fluid contents include the actuating bag 220 and the piezoelectric device 230 that are disposed in the cavity 211. In examples where the housing 210 is made of a fabric or fabric-like material with a degree of porosity that permits air or even a liquid to pass through the wall(s) 212/216 of the housing 210, the cavity 211 may have a variable volume. Even in the embodiments where the cavity 211 has a variable volume, the housing 210 limits the actuating bag 220 from increasing beyond a maximum size. In this sense, in some embodiments, the cavity 211 may alternatively be described as having a “fixed volume”. In the present disclosure, the term “fixed volume” refers to the housing 210 having limited ability to stretch or change in size, such that for practical purposes (disregarding effects of compression, pressure, and/or temperature, etc.), the cavity 211 can be regarded as having an unchanged volume or size. The term “fixed volume” may also be understood to mean that the overall size of the housing 210 is not significantly increased by an inflation of the actuating bag 220. In some embodiments, the cavity 211 may alternatively be configured as a hermetically sealed cavity in some examples where a substantially nonporous or waterproof material is used for the housing 210. In other examples, such as that illustrated by FIG. 2, the cavity 211 is not hermetically sealed but is in fluid communication with the environment external of the housing 210 via a pneumatic passage 204 and a pneumatic port 204 and/or via a signal port 206.

In some examples, the housing 210 may be formed as one integral unit. Alternatively, the constraining wall 212 and the flexible wall 214 may be formed as two or more parts and assembled together.

The sensor 200 includes a piezoelectric device 230 which is disposed in the cavity 211 adjacent the flexible wall 214. The piezoelectric device 230 may be coupled to an interior face 214b of the flexible wall 214, i.e., the surface defining or facing the cavity 211. The piezoelectric device 230 may be a polymer-based device. For example, the piezoelectric device 230 may be a poly(vinylidene fluoride)-based (PVDF-based) piezoelectric nanogenerator (PENG). The piezoelectric device 230 is configured to be flexible and deformable. Preferably, the elasticity of the piezoelectric device 230 is configured to match the elasticity of the flexible wall 214, e.g., having an elasticity that is about the same as or higher than an elasticity of the flexible wall 214. If the piezoelectric device 230 is pressed or pushed against the interior face 214b of the flexible wall 214, the piezoelectric device 230 deforms, stretches, or changes in shape as the piezoelectric device 230 conforms with the flexible wall 214. The thickness or material properties of each of the piezoelectric film 232 and the conductive layers 234/236 may be configured to mitigate potential delamination between these layers.

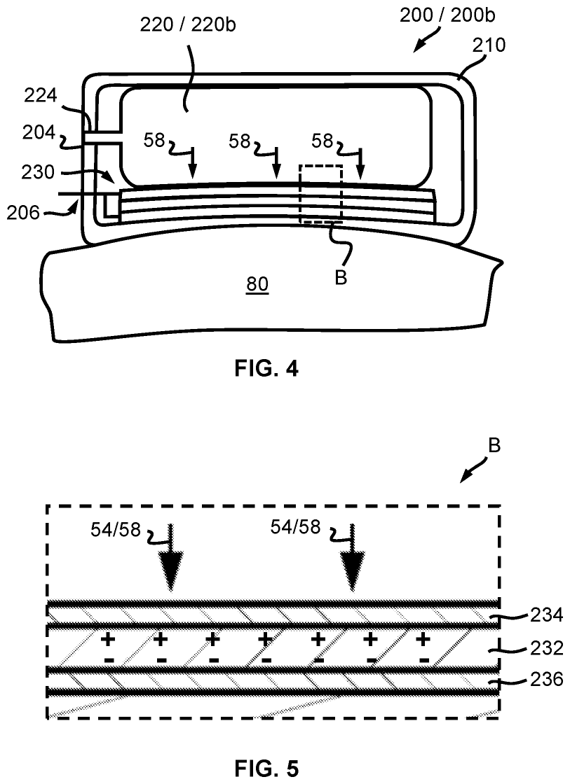

As illustrated by the close-up view A in FIG. 3, the piezoelectric device 230 may be configured as a layered structure, in which a piezoelectric film 232 is sandwiched between two conductive layers (a first conducting layer 234 and a second conducting layer 236). The piezoelectric device 230 is disposed with the second conductive layer 236 immediately adjacent the interior face 214b of the flexible wall 214. In some examples, the piezoelectric device 230 may extend over substantially all or most of the interior face 214a. In other examples, the piezoelectric device 230 may be substantially centrally disposed relative to the interior face 214a. Making reference to FIG. 4 and FIG. 5, in use, the piezoelectric device 230 is configured to receive a compressive force 58, and to generate a voltage in response. The compressive forces are generally provided by the actuating bag 220 along a direction 54 that is substantially normal to a contact area between the actuating bag 220 and the first conductive layer 234 of the piezoelectric device 230. The potential difference or voltage generated across the first conductive layer 234 and the second conductive layer 236 is due to a change in the dipole moment in the piezoelectric film 232, such that electrical charges are accumulated at the respective conducting layers 234/236, i.e., a potential difference is established across the piezoelectric film 232. The resulting signal corresponding to the voltage formed can then be acquired via a signal port 206.

The sensor 200 includes an actuating bag 220 which will be described with the aid of FIG. 2 and FIG. 4. The actuating bag 220 is disposed in the cavity 211 between the piezoelectric device 230 and the constraining wall 212. More specifically, the actuating bag 220 and the interior face 214b of the flexible wall 214 are configured to contact substantially opposing sides of the piezoelectric device 230. As illustrated by FIG. 2 and FIG. 4, the actuating bag 220 has a variable bag volume. Optionally, the actuating bag 220 is flexible, and may deform or stretch elastically. The actuating bag 220 is configured to inflate and/or deflate such that the variable bag volume is changeable between an initial volume 220a (FIG. 2) and a threshold volume 220b (FIG. 4). Air or a suitable actuating fluid may be received by or expelled from the actuating bag 200 via a pneumatic passage 224 and through a pneumatic port 204. The housing 210 may be configured such that the actuating bag 220 is constrainable by the housing 210 when the actuating bag 220 is in an inflated state. The actuating bag 220 may be considered to be in an inflated state (as opposed to being in a deflated state) if there is some air or actuating fluid in the actuating bag 220. The actuating bag 220 in the inflated state may be fully inflated or partially inflated. The actuating bag 220 and/or the cavity 211 may be sized and/or shaped, or formed of one or more materials, such that the actuating bag 220 in an inflated state is constrainable by the housing 210 from further inflating beyond a certain size or volume.

FIG. 2 illustrates the sensor 200 in an initial state 200a, in which the actuating bag 220 is configured with an initial volume 220a. The initial volume 220a may be one in which the actuating bag 220 is deflated or not fully inflated. In the initial state 200a, the actuating bag 220 does not exert a significant compressive force or pressure on the piezoelectric device 230. In some examples, in the initial state 200a, the actuating bag 220 does not contact the piezoelectric device 230. In some examples, in the initial state 200a, the actuating bag 220 may contact the piezoelectric device 230 but provides a negligible force on the piezoelectric device 220.

FIG. 4 illustrates the sensor 200 in an inflated state or a measurement state 200b, in which the actuating bag 220 is inflated to a threshold volume 220b. The threshold volume 220b is larger than the initial volume 220a. The threshold volume 220b of the actuating bag 220 may be defined as the maximum volume to which the variable bag volume can increase within the confines of the housing 210 or cavity 211. Alternatively described, the variable bag volume is at the threshold volume 220b if the actuating bag 220 is inflated until the variable bag volume is constrained by the housing 210 from further expanding or inflating. In some examples, the variable bag volume is at the threshold volume 220b if the actuating bag 220 is inflated until the variable bag volume is constrained by the constraining wall 212. In some examples, the actuating bag 220 is pneumatically operable to increase its variable bag volume until the actuating bag 220 applies a pressure or compressive force on the piezoelectric device 230 such that a signal is obtainable from the piezoelectric device 230, i.e., from the sensor 200. The actuating bag 220 may be described as being constrainable by the housing 210 to press the flexible wall 214 against a body part of the user.

Referring again to FIG. 2, in the initial state 200a, the sensor 200 may be attached to or worn by a subject, with the exterior face 214a of the flexible wall 214 in abutment with a body part of the subject. The sensor 200 may be disposed on the subject's skin or body part 80 near the muscle of interest. Since the body part may be rounded or somewhat irregularly shaped, the flexible wall 214 of the sensor 200 in the initial state 200a may not be completely compliant with the shape of the body part. In the example shown in FIG. 2, the sensor 200 is secured or attached to the body part 80, but there may be a gap between parts of the flexible wall 214 and the body part.

Referring now to FIG. 4, in the measurement state 200b, the actuating bag 220 is inflated and pressing against the constraining wall 212 and the piezoelectric device 230. The actuating bag 220 in effect applies a compressive force 58 directly on the piezoelectric device 230 and indirectly on the flexible wall 214, such that the flexible wall 214 is compressed between the device 200 and the body part 80. That is, in the measurement state, the actuating bag 220 is in abutment with the constraining wall 212 and the piezoelectric device 230. The flexible wall 214 is in abutment with the body part 80 when the device 200 is in the measurement state 200b. As illustrated, the flexible wall 214 is in compliant contact with the body part 80. That is, the actuating bag 200 is configured to be pneumatically operable to press an exterior face 214a of the flexible wall 214 against the body part 80. If there is a certain level of muscle stiffness at the body part (e.g., in the muscle under the sensor 200), the actuating bag 220 and the body part 80 combine to subject the piezoelectric device 230 to a combination of pressure and strain changes. The resulting signal corresponds to a measurement of a level of muscle stiffness at the body part. In other words, the level of muscle spasticity or stiffness can be measured in a quantitative and repeatable manner by the sensor 200.

The measurement state 200b may be defined as a state in which a further increase in the pressure and/or the volume of the actuator bag 220 does not yield significant changes in the compressive force 58 applied to the piezoelectric device 230. In some examples, the measurement state 200b is defined by a threshold volume of the actuating bag 220 in which the actuating bag 220 is sustained or held at a substantially constant volume over a measurement duration. In other examples, the measurement state 200b is defined by a threshold pressure of the actuating bag 220 in which the actuating bag 220 is sustained or held at a substantially constant pressure over a measurement duration. The measurement duration refers to a period of time when the signals are acquired and a quantitative measure of a level of muscle spasticity is determined. The measurement duration may be as short as a few seconds or a few minutes.

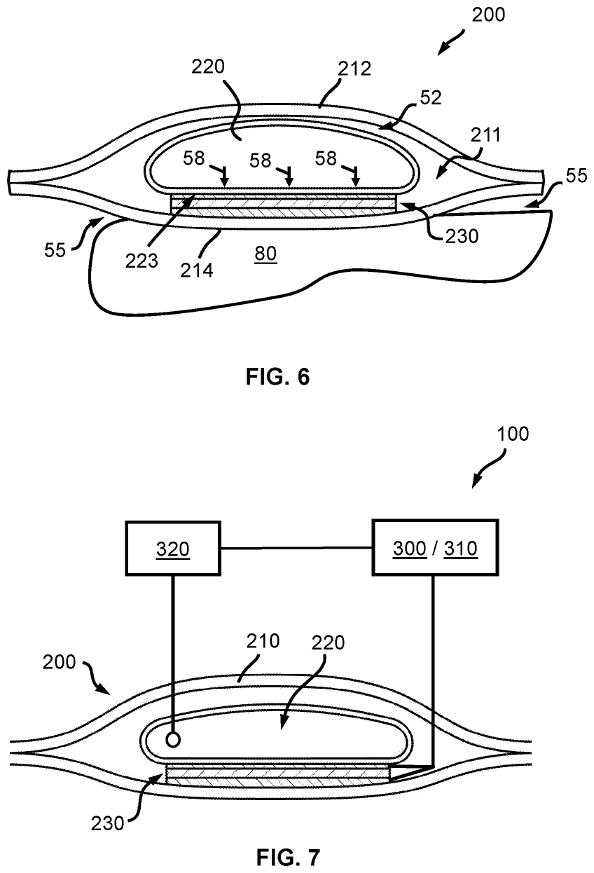

FIG. 6 is a schematic cross-sectional drawing showing another embodiment of the sensor 200 in the measurement state 200b. In this example, the sensor 200 is disposed at a body part 80 where there is a significant curvature of the body part and there may be gaps 55 (exaggerated for the purpose of illustration) between the sensor 200 and the body part 80 when the sensor 200 is in the measurement state. In some embodiments of the sensor 200, in the measurement state 200b, there may be a gap 52 (exaggerated for the purpose of illustration) between part of the actuating bag 220 and the constraining wall 212. The sensor 200 remains operable despite the presence of such gaps 52/55, with the actuating bag 220 being configurable to constrained by the housing 210 and/or the cavity 211 to apply compressive forces 58 on the piezoelectric device 230, in which the piezoelectric device 230 is concurrently conformably compressed between the flexible wall 214 and the actuating bag 220.

Optionally, in the initial state 200a, as illustrated in FIG. 1 and FIG. 2, the actuating bag 220 may be disposed spaced apart from the piezoelectric device 230 to form a first gap 50 therebetween. That is, in the initial state 200a, the actuating bag 220 may be disposed such that the actuating bag 220 is not in contact with the piezoelectric device 230. Optionally, the actuating bag 220 may also be spaced apart from the constraining wall 212 such that there is a second gap 52 therebetween. The subject is free to move about without fear that the sensor 200 will give false readings. Optionally, as illustrated in FIG. 6, the actuating bag 220 may be attached to the piezoelectric device 230 to ensure an area of contact between the actuator bag 220 and the piezoelectric device 230.

Optionally, the actuating bag 220 may be shaped to provide a first abutment surface 223 to facilitate a larger potential contact area with the piezoelectric device 230. The first abutment surface 220a is pliant to promote conformance of the first abutment surface with to the piezoelectric device 230, and to apply compressive forces over as much of the piezoelectric device 230 as possible. Alternatively, the first abutment surface 220a may be configured with a higher rigidity in comparison to other portions of the actuating bag 220, such that when the variable bag volume 222 is increased, the first abutment surface 220a remains substantially flat to apply compressive forces on the piezoelectric device 230 over as large an area as possible.

FIG. 7 illustrates an embodiment of a muscle spasticity measurement system 100 according to one embodiment of the present disclosure. The muscle spasticity measurement system 100 includes the sensor 200 and a controller 300. The controller 300 is operable coupled to the sensor 200. The controller 200 may include a signal communication module or a data acquisition device 310 configured to receive signals from the sensor 200. The muscle spasticity measuring system 100 includes a pneumatic source 320. The pneumatic source 320 may be controlled by the controller 300 such that air or another actuating fluid may be delivered from the pneumatic source 320 to inflate the actuating bag 220 and to deflate the actuating bag 220, and thereby enable a variable bag volume. The controller 300 may be configured to inflate or pressurize the actuating bag 220 and to acquire a signal from the piezoelectric device 230 when the sensor 200 is in the measurement state 200b. The controller 300 may be configured to output the signal as a muscle stiffness value corresponding to a level of muscle stiffness. This may be iteratively performed over time to acquire a plurality of the signals for the same muscle. That is, the muscle spasticity measurement system 100 is configured to provide a non-invasive method for sensing muscle spasticity and/or providing quantitative and repeatable measurements of the level of muscle stiffness of a body part 80 of a subject 82.

In some embodiments, the signal is received over the measurement duration, for example a few seconds, such that the signal is in the form of a waveform or wavelet. Further, the controller 300 may be configured to determine a muscle stiffness value (a quantitative measure of muscle stiffness) based on the waveform or wavelet. In some embodiments, the sensor 200 may be iteratively switched between the initial state 200a and to the measurement state 200b, and the controller 300 may be configured to acquire a plurality of muscle stiffness values and/or determine a mean muscle stiffness value.

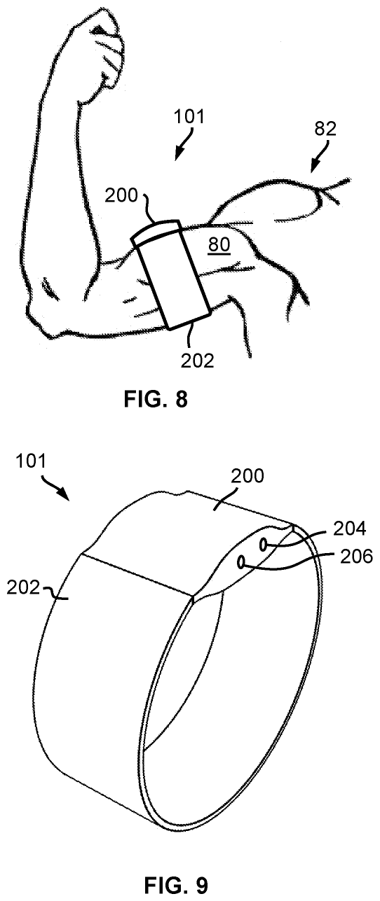

FIGS. 8 and 9 illustrate a muscle spasticity measurement wearable product 101 that includes a SPASM device 200 (sensor 200) that may be detachably coupled with an attachment 202. In this example, the body part 80 may be a bicep muscle of a subject 82, as illustrated. In some embodiments, the attachment 202 may be configured to be wearable on different body parts of the subject 82, for example bicep, thigh, calves, etc. As a non-limiting example, the attachment 202 includes a re-sizeable band for wrapping around a bicep 80 of the subject 82 and holding the sensor 200 in place relative to the bicep. In other examples, the attachment 202 may include a Velcro attachment for securing the sensor 200 to the bicep.

The controller 300 may be configured for enabling signal communication with the sensor 200. As an example, the controller 300 may be configured to receive a signal from the sensor 200 and to determine a level of muscle stiffness based on the signal. Further, the system 100 may be provided with a display for displaying the level of muscle stiffness and/or a representation of the level of muscle stiffness. The level of muscle stiffness may be represented as a mean signal value or in relative terms, such as whether the muscle stiffness is below or above a measurement threshold, etc. The wearable product 101 may include a controller 300 that is directly coupled to the sensor 200. Alternatively, the controller 300 may be coupled to a signal port 206 of the sensor 200, in a wired coupling or wirelessly. A pneumatic source 320 may be coupled to a pneumatic port 204 of the sensor 200.

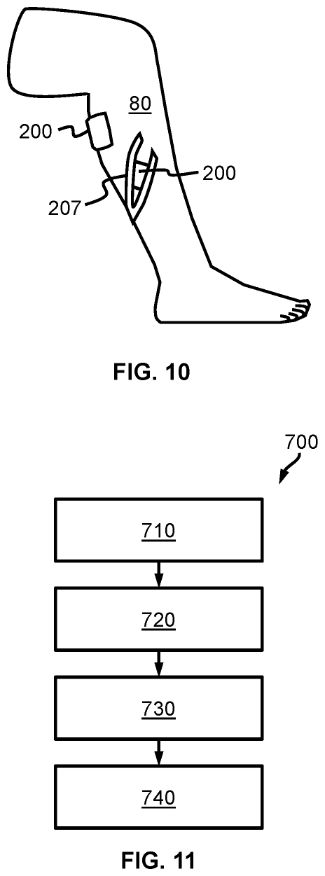

The sensor 200 and muscle spasticity measurement system 100 may be attached or worn at various places and orientations. In FIG. 10, one example of the sensor 200 includes a self-adhesive external face of the flexible wall 214 so that the sensor may be attached to the relevant body part without the use of an attachment 202. FIG. 10 also illustrates another example of the sensor 200 which is held in place at a body part with the aid of adhesive medical tape or sports tape 207. The portability of the sensor 200 enables a level of muscle stiffness to be measured in a quantitative and repeatable manner, in a broader range of applications, with minimal discomfort to the subject.

FIG. 11 shows a flowchart of a method 700 of measuring muscle spasticity at a body part. For the sake of convenience, the method 700 is illustrated in blocks 710, 720, 730, 740. It will be understood from the above description that some of the blocks may not strictly follow a sequence in which one block begins upon completion of an immediately preceding block. That is, the blocks represent events or steps that may occur concurrently, and that the method 700 may not be easily divided into discrete steps. The method 700 may include attaching the sensor 200 to a body part (710) with the flexible wall 214 near a target muscle to be measured. The method 700 includes providing a compressive force on the piezoelectric device 230, by increasing the variable bag volume of the actuating bag 220 until further increase in the variable bag volume is limited by the housing 210 (720). In the measurement state 200b, the actuating bag 220 is at least partially in abutment with the constraining wall 212, with the actuating bag 220 pressing on one of the two contacts of the piezoelectric device 230 (730). The method 200 includes determining a quantitative measure of a level of muscle stiffness (muscle stiffness value) at a body part (740), based on a signal acquired from the piezoelectric device 230 in the measurement state 200b of the sensor 200. The method 700 may further include expanding the actuating bag 220 to a threshold volume; and holding the actuating bag 220 at the threshold volume. Alternatively, or additionally, the method 700 further includes expanding the actuating bag 220 to a threshold pressure; and holding the actuating bag 220 at the threshold pressure. Further, the signal may be received over time to determine a muscle stiffness value. The method 700 may further include iteratively increasing and decreasing the variable bag volume of the actuating bag 220; and measuring a respective plurality of signals. Alternatively, or additionally, the method 700 may further include iteratively increasing and decreasing a pressure in the actuating bag 220; and measuring a respective plurality of signals.

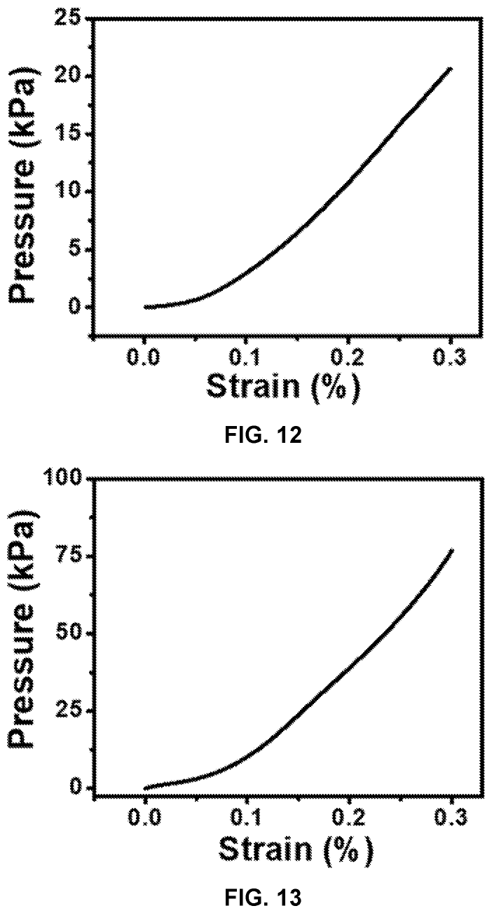

In one set of experiments, to demonstrate the workability of the muscle spasticity measurement system 100, tests were performed on porcine limb muscle to verify the proposed sensor and method of muscle stiffness measurement. To emulate the conditions of a stiff muscle, one sample of porcine muscle was kept at room temperature and another sample of frozen porcine muscle which had been kept under freezing conditions at minus 20° C. (20 degrees Celsius below zero) for one hour. A conventional compressive mechanical stress-strain experiment under a controlled laboratory environment was first performed with a strain rate of 10 millimeters/minute. FIG. 12 shows a stress-strain plot of the porcine muscle kept at room temperature, which is characterized by a lower muscle stiffness value (Young's Modulus of Elasticity) of 68 kPa. FIG. 13 shows a stress-strain plot of the frozen porcine muscle (kept at minus 20° C. for one hour), which is characterized by a higher muscle stiffness value (Young's Modulus of Elasticity) of 253 kPa. The conventional stress-strain method is a destructive test and cannot be used on patients.

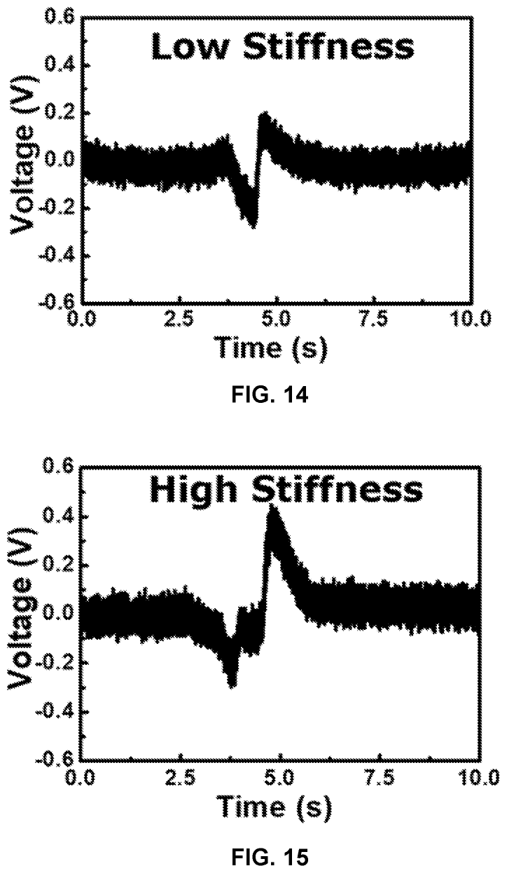

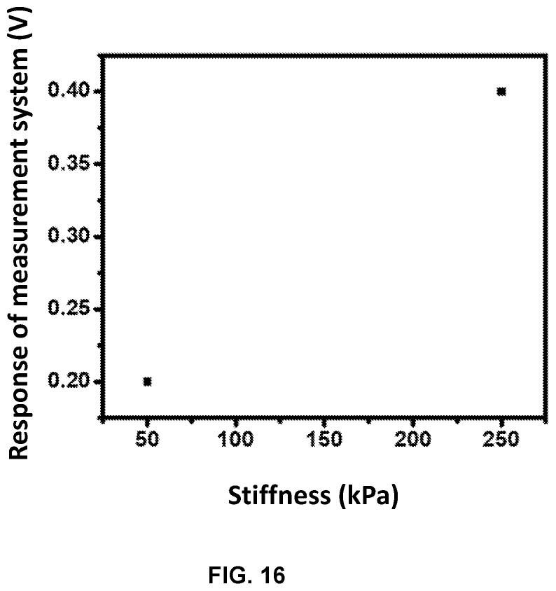

The muscle spasticity measurement system 100 was also deployed in measuring the two types of porcine muscles. The actuating bag 220 was inflated or pressurized at a pressure of 40 mmHg in the measurement state. FIG. 14 shows a voltage response (in wavelet form) of the signal received from the muscle spasticity measurement system 100 for the sample of porcine muscle kept at room temperature. FIG. 15 shows a voltage response (in wavelet form) of the signal received from the muscle spasticity measurement system 100 for the sample of frozen porcine muscle. It can be observed that the voltage peak corresponds to the stiffness of the respective porcine muscle, and provides a means of differentiating between porcine muscle with different muscle stiffness values. This demonstrates the effectiveness of the proposed muscle spasticity measurement system 100 for determining various levels of muscle stiffness. FIG. 16 shows the response of the sensor 200 as a function of the stiffness of the porcine muscle. The experiments demonstrate the viability of the sensor 200 and the muscle spasticity measurement system 100 for providing a repeatable method of obtaining a quantitative measure of muscle spasticity (muscle stiffness values).

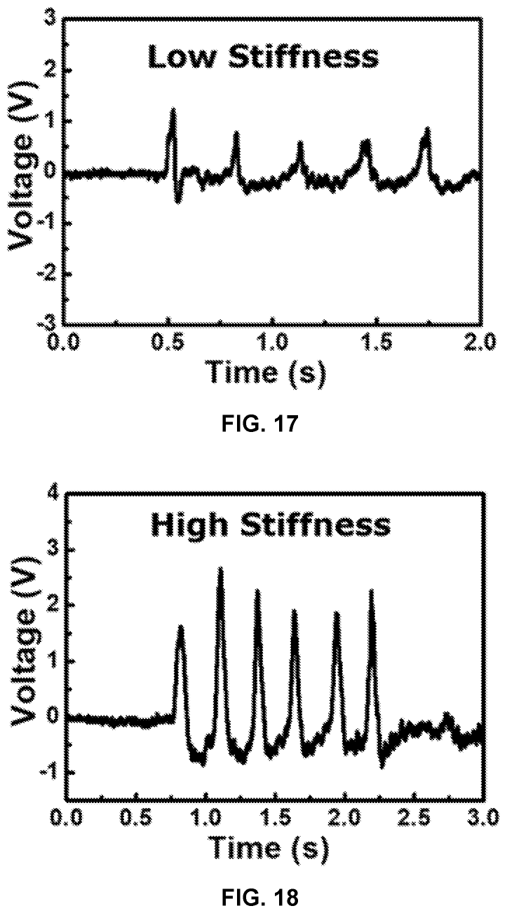

In another example of practical application, the proposed muscle spasticity measurement system 100 was used to measure muscle spasticity of a human subject in a set of experiments. The sensor 200 was attached to the bicep muscle of the subject. To demonstrate the workability of the sensor 200 for low levels of muscle stiffness, the measurements were carried out on the bicep muscle without straining the bicep muscle. To demonstrate the workability for high muscle stiffness, the measurements were carried out on muscle under artificially tightening or with strengthening of the muscle. FIG. 17 shows a voltage response (in wavelet form) of the signal received from the muscle spasticity measurement system 100 for measuring the bicep muscle with low muscle stiffness. FIG. 18 shows a voltage response (in wavelet form) of the signal received from the muscle spasticity measurement system 100 for measuring the bicep muscle with high muscle stiffness. It may be observed that the measurements were iteratively performed with the voltage response quantitatively and qualitatively differentiable between human muscle of different spasticity. This demonstrates the effectiveness of the proposed muscle spasticity measurement system for determining or measuring various levels of muscle spasticity.

The sensor 200 and the muscle spasticity measurement system 100 proposed herein are non-invasive in nature and can be used on patients/subjects without causing any damage to the body part that is measured. This allows the measurements to be done iteratively to obtain a mean stiffness value and thus be more accurate than conventional methods. In other words, the controller of the muscle spasticity measurement system may be configured to acquire a plurality of the signal over time, and to determine a mean muscle stiffness value at the body part based on the plurality of the signal. The measurements may be done with minimal disturbances, e.g., to measure different body parts or different conditions of the muscle, all that is required is a simple change in the location/position of sensor 200 or a change in the position or posture of the body part 80, etc. Portability of the muscle spasticity measurement system 100 allows muscle spasticity to be measured in various practical conditions, such as with the subject on a hospital bed, at home, or at an incident site. This alleviates the need to move or transport the subject to a specific test environment, such as a hospital or laboratory. This is advantageous for patients suffering from stroke, cerebral palsy, or spinal cord injury, as travelling can be challenging and time-consuming for such subjects. Further, the setup of the muscle spasticity measurement system 100 requires relatively few parts or components, and quick and easy deployment is possible without the need for laborious setup or heavy machineries. This is advantageous in times of emergency where any delay in measurement may adversely affect the patient's well-being, but where any early data may aid accurate diagnosis of the patient's condition. The sensor 200 is also configurable for a wide range and versatility in attachments 202 to suit the use of the sensor 200 at different body parts.

All examples described herein, whether of apparatus, methods, materials, or products, are presented for the purpose of illustration and to aid understanding, and are not intended to be limiting or exhaustive. Various changes and modifications may be made by one of ordinary skill in the art without departing from the scope of the invention as claimed.

1. A sensor attachable to a subject, the sensor comprising:
a housing having a flexible wall and a constraining wall, the housing defining a cavity;
an actuating bag disposed in the cavity; and
a piezoelectric device disposed in the cavity between the actuating bag and the flexible wall, the piezoelectric device being coupled to the housing substantially via an interior face of the flexible wall, wherein the actuating bag is configured to be pneumatically operable, and wherein the actuating bag in an inflated state is constrainable by the housing to press an exterior face of the flexible wall against a body part of the subject.

2. The sensor as recited in claim 1, wherein the cavity is characterized by a fixed volume.

3. The sensor as recited in claim 1, wherein the cavity is characterized by a closed volume.

4. The sensor as recited in claim 1, wherein the sensor is configured to measure a level of muscle stiffness at the body part.

5. The sensor as recited in claim 4, wherein the level of muscle stiffness corresponds to a muscle stiffness of a bicep of the subject.

6. The sensor as recited in claim 1, wherein the actuating bag is characterized by a variable bag volume, the actuating bag being pneumatically operable to increase in the variable bag volume until the variable bag volume is constrained by the housing.

7. The sensor as recited in claim 1, wherein the actuating bag is pneumatically operable to expand until the actuating bag is constrained by the constraining wall.

8. The sensor as recited in claim 1, wherein the flexible wall comprises a first surface and a substantially opposing second surface, and wherein the second surface is a part of an external surface of the sensor.

9. The sensor as recited in claim 1, wherein the flexible wall is resiliently deformable to vary a shape of the cavity.

10. The sensor as recited in claim 1, wherein the piezoelectric device is configured to provide a signal corresponding to a measurement state of the sensor, and wherein in the measurement state the actuating bag is in abutment with the constraining wall and the piezoelectric device.

11. The sensor as recited in claim 10, wherein the flexible wall is in abutment with the body part.

12. The sensor as recited in claim 10, wherein the actuating bag is configured to expand to a threshold pressure and wherein the actuating bag is configured to sustain a substantially constant pressure in the measurement state.

13. The sensor as recited in claim 10, wherein the actuating bag is configured to expand to a threshold volume and wherein the actuating bag is configured to sustain a substantially constant volume in the measurement state.

14. (canceled)

15. A muscle spasticity measurement system comprising:
the sensor as recited in claim 1; and
a controller operably coupled to the sensor, the controller being configured to receive a signal from the piezoelectric device and to determine a muscle stiffness value based on the signal.

16. The muscle spasticity measurement system as recited in claim 15, wherein the controller is configured to controllably expand the actuating bag in the cavity.

17. The muscle spasticity measurement system as recited in claim 16, wherein the controller is configured to:
expand the actuating bag to a threshold pressure; and
hold the actuating bag at the threshold pressure.

18. The muscle spasticity measurement system as recited in claim 17, wherein the controller is further configured to:
receive the signal produced by the piezoelectric device when the actuating bag is at the threshold pressure; and
determine the muscle stiffness value based on the signal.

19. The muscle spasticity measurement system as recited in claim 16, wherein the controller is configured to:
expand the actuating bag to a threshold volume; and
hold the actuating bag at the threshold volume.

20. The muscle spasticity measurement system as recited in claim 19, wherein the controller is further configured to:
receive the signal produced by the piezoelectric device when the actuating bag is at the threshold volume; and
determine the muscle stiffness value based on the signal.

21. The muscle spasticity measurement system as recited in claim 15, wherein controller is configured to acquire a plurality of the signal is received over time, and wherein the controller is configured to determine a mean muscle stiffness value at the body part based on the plurality of the signal.
